SEQUENCE LISTING (4 sequences)

SEQ ID NO: 1 - DNA, 20 bp, Artificial Sequence
  Oligonucleotide primer
acaactcgttgtgccaagcc

SEQ ID NO: 2 - DNA, 20 bp, Artificial Sequence
  Oligonucleotide primer
gcatcgcatagaggttgcgc

SEQ ID NO: 3 - DNA, 22 bp, Artificial Sequence
  Oligonucleotide primer
ctacaatgtgctgctcctgaac

SEQ ID NO: 4 - DNA, 20 bp, Artificial Sequence
  Oligonucleotide primer
caggtgagcacgtagagcac

(19) United States
(12) Patent Application Publication
(10) Pub. No.: US 2004/0185509 A1
(43) Pub. Date: Sep. 23, 2004

(54) Eosinophil prostaglandin d2 receptor assays

(76) Inventors: Francois Gervais, Pierrefonds (CA); Francois Nantel, Sherbrooke (CA)

Correspondence Address: MERCK AND CO INC, P O BOX 2000, RAHWAY, NJ, 070650907

(21) Appl. No.: 10/483,914

(22) PCT Filed: Jul. 17, 2002
(86) PCT No.: PCT/CA02/01112

Related U.S. Application Data
(60) Provisional application No. 60/306,357, filed on Jul. 18, 2001.

(51) Int. Cl. G01N 33/53; G01N 33/567

(52) U.S. Cl. 435/7.21

(57) ABSTRACT

The present invention identifies different activities mediated by eosinophil PGD2 receptors and features methods measuring the ability of a compound to modulate such activities. Activities mediated by eosinophil PGD2 receptors include those associated with CRHT2 and those associated with the DP receptor. Activities identified herein as associated with eosinophil CRHT2 include a change in cell morphology, degranulation, and a specific chemokinetic effect. Activities identified herein as associated with the eosinophil DP receptor include resistance to apoptosis.

### CROSS-REFERENCE TO RELATED APPLICATIONS

The present application claims priority to provisional application U.S. Serial No. 60/306,357, filed Jul. 18, 2001, hereby incorporated by reference herein.

### BACKGROUND OF THE INVENTION

The Background of the Invention and references cited in the present application are not admitted to be prior art to the claimed invention.

Prostanglandin D₂ (PGD₂) is a cyclooxygenase metabolite of arachidonic acid. (Narumiya, et al., Physiological Reviews 79:1193-1226, 1999.) PGD₂ has been implicated in playing a role in different physiological events such as sleep and allergic responses. (Boie, et al., The Journal of Biological Chemistry, 270:18910-18916, 1995, Narumiya, et al., Physiological Reviews 79:1193-1226, 1999, Matsuoka, et al., Science 287:2013-2017, 2000.)

Mast cells and TH2 cells are important immune cells involved in allergic responses. PGD₂ is released from mast and TH2 cells in response to an immunological challenge. (Roberts, et al., N. Engl. J. Med. 303:1400, 1980, Lewis, et al., J. Immunol. 129:1627, 1982, Tanaka, et al., J. Immunol. 164:2277, 2000.)

Receptors for PGD₂ include the “DP” receptor, the chemoattractant receptor-homologous molecule expressed on TH2 cells (“CRTH12”), and the “FP” receptor. These receptors are G-protein coupled receptors activated by PGD₂. PGD₂ is a non-selective agonist at the FP receptor. (Abramovitz, et al., Biochimica et Biophysica Acta 1483:285-293, 2000.)

Abramovitz, et al., U.S. Pat. No. 5,958,723 and Boie, et al., Journal of Biological Chemistry 270:18910-18916, 1995, describe the cloning and characterization of the human DP receptor. These references also indicate that PGD₂ activates the DP receptor.

Abe, et al., Gene 227:71-77, 1999, Nagata, et al., FEBS Letters 459:195-199, 1999, and Nagata, et al., The Journal of Immunology 162:1278-1286, 1999, describe CRTH2 and its expression on different cells including human T-helper cells, basophils, and eosinophils. Hirai, et al., J. Exp. Med. 193:255-261, 2001, indicates that CRTH2 is a receptor for PGD₂.

### SUMMARY OF THE INVENTION

The present invention identifies different activities mediated by eosinophil PGD₂ receptors and features methods measuring the ability of a compound to modulate such activities. Activities mediated by eosinophil PGD₂ receptors include those associated with CRHT2 and those associated with the DP receptor. Activities identified herein as associated with eosinophil CRHT2 include a change in cell morphology, degranulation, and a specific chemokinetic effect. Activities identified herein as associated with the eosinophil DP receptor include resistance to apoptosis.

Measuring the ability of a compound to modulate a PGD₂ receptor activity can be performed quantitatively or qualitatively. Compounds modulating PGD₂ receptor activity include agonists, antagonists and allosteric modulators.

Thus, a first aspect of the present invention features a method that measures the effect of a test compound on either apoptosis or degranulation as a measure of the ability of the compound to modulate a PGD₂ receptor activity. The method employs eosinophil cells.

Another aspect of the present invention describes a method of assaying the ability of a test compound to modulate CRTH2 activity using a compound identified as binding to CRTH2. The method comprises the steps of: (a) identifying a compound that binds to human CRTH2; (b) providing the compound to an eosinophil; and (c) measuring eosinophil morphology, chemokinesis under conditions distinguishing chemokinesis from chemotactic ability, or degranulation.

Another aspect of the present invention describes a method of assaying the ability of a test compound to modulate CRTH2 activity involving the use of a CRTH2 agonist. The method comprises the steps of: (a) providing the test compound and an CRTH2 agonist to an eosinophil, and (b) measuring either eosinophil morphology, chemokinesis under conditions distinguishing chemokinesis from chemotactic ability, or degranulation.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates PGD₂ receptor expression on human eosinophils. RT-PCR was performed on total RNA isolated from purified human eosinophils using CRTH2 or DP-specific primers. The RT-PCR product was revealed, by Southern blot, using a CRTH2-specific (top panel) or DP-specific (lower panel) radioactive probe. RNA from HEK cells expressing recombinant CRTH2 or DP receptor was used as a positive control in lane 1. RT-PCR using 18S ribosomal RNA-specific primers was conducted in parrallel to ensure that equivalent amounts of RNA were used between each donor (data not shown). The bands seen are derived from mRNA and not genomic DNA since no signal is detected in absence of reverse transcriptase. Results from two out of four donors tested are shown.

FIG. 2 illustrates a rapid change in eosinophil morphology induced by PGD₂. Purified human eosinophils were incubated for 15 minutes with various agents in a 24-well dish. Cells were then magnified 200-times using an inverted microscope. a, vehicle-treated eosinophils. b, eosinophils treated with 10 nM PGD₂. c, 1 μM BW245C (a DP-selective agonist) d, 10 nM 13,14-dihydro-15-keto-PGD₂ (DK-PGD₂). e, 100 nM platelet-activating factor; PAF. f, 1 ng/ml of interleukin-5; IL-5. A representative experiment from 20 donors tested is shown.

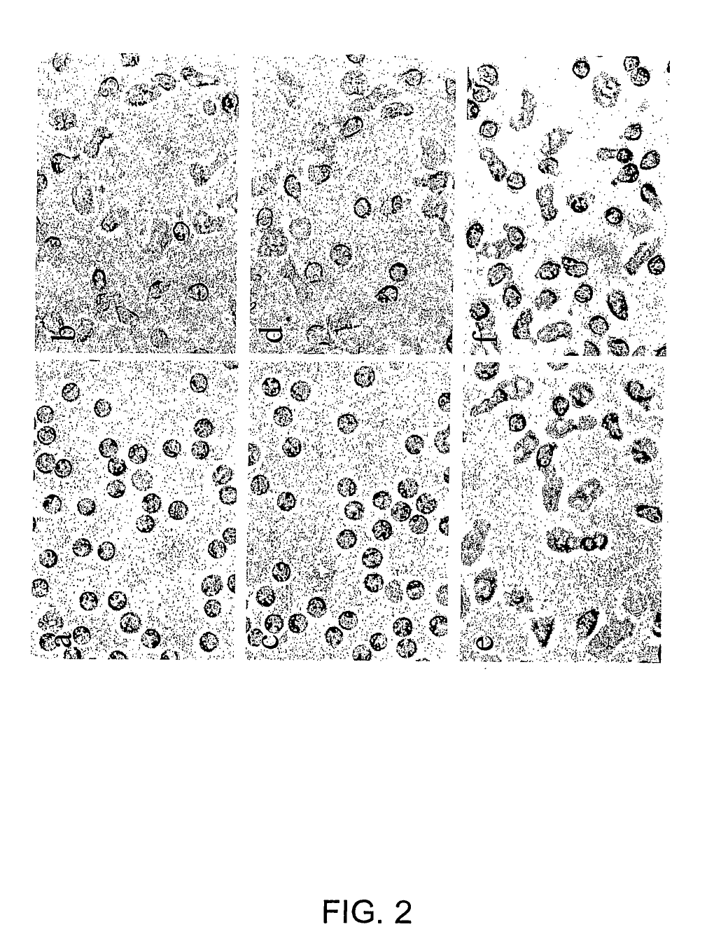

FIG. 3 illustrates the effect of PGD₂ on eosinophil chemokinesis. Purified human eosinophils were treated for 5 minutes with various agents prior to being placed in the upper chamber of a chemotactic unit. No chemoattractant was added to the lower chamber in order to simply measure chemokinesis. After two hours, the number of cells that transmigrated to the lower chamber was evaluated with an hematocytometer. Chemokinesis efficiency is expressed as the number of transmigrating cells with the agent divided by the number of transmigrating cells with vehicle only (fold-increase chemokinesis over background). Lane 1, vehicle treated eosinophils. Lane 2, eosinophils were treated with 100 nM PGD₂, lane 3 with 1 μM BW245C, lane 4 with 100 nM DK-PGD₂, lane 5 with 100 nM of platelet activating factor, lane 6 with 1 ng/ml of interleukin-5 and lanes 7-8-9 with 1 μM of the indicated compounds. For each experiment, each condition was tested in two independent wells. The mean response is indicated by a dash. The effect of PGD₂ at 100 nM is significant with a probability of <0.001 in repeated measures ANOVA followed by paired t-tests.

FIG. 4 illustrates the ability of PGD₂ to trigger eosinophil degranulation. Purified human eosinophils were treated for 1 hour with various agents. The amount of ECP released in the media was then determined by radioimmunoassay. Lane 1, vehicle treated cells. Lane 2, eosinophils were treated with 100 nM PGD₂, lane 3 with 1 μM BW245C, lane 4 with 100 nM DK-PGD₂, lane 5 with 100 nM of platelet activating factor, lane 6 with 1 ng/ml of interleukin-5 and lanes 7-8-9 with 1 μM of the indicated compounds. The values correspond to the amount of ECP detected under the various conditions minus the value obtained with the vehicle treated cells (amount of ECP over background). For each experiment, each condition was tested in duplicate. The mean response is indicated by a dash. The effect of PGD₂ at 100 nM was tested on 11 donors (p<0.0003 in t-test) while the effect of DK-PGD₂ at 100 nM was tested on 8 donors (p<0.01). The value presented for PAF is the mean of six independent experiments (p<0.01).

FIG. 5 illustrates the ability of PGD₂ to increase the survival of eosinophils in culture. Purified human eosinophils were maintained in culture in the presence of various agents for 36 hours. The cells were then harvested and the extent of apoptosis was evaluated by flow cytometry (Annexin V/propidium iodide staining). Cells that have not reached the stage of late apoptosis (thus not positive for both annexin V and propidium iodide staining) were considered to be alive. Lane 1, vehicle treated cells. Lane 2-9, eosinophils treated with 1 μM of the indicated compounds except lane 6 where interleukin-5 was used at 1 ng/ml. The values correspond to the percentage of non-late apoptotic eosinophils in the treated population minus the percentage of non-late apoptotic eosinophils in the vehicle treated population. The mean response is indicated by a dash. The effect of PGD₂ at 1 μM was tested on 7 donors (p<0.1 in t-test) while the effect of BW245C at 1 μM was tested on 9 donors (p<0.0005).

### DETAILED DESCRIPTION OF THE INVETION

Identifying different effects mediated by eosinophil PGD₂ receptor activation provides for indicators that may be measured to evaluate the ability of a compound to modulate eosinophil PGD₂ receptor activity and provides information concerning the physiological effects of PGD₂ receptor activation. Information concerning the physiological effects of PGD₂ receptor activation can be used to help evaluate the importance of inhibiting a PGD₂ receptor activity.

Compounds modulating eosinophil PGD₂ receptor activity have a variety of different uses including utility as a tool to further study PGD₂ receptor activity and as an agent to achieve a beneficial effect in a patient. Modulating PGD₂ receptor activity includes evoking a response at the receptor and altering a response evoked by a PGD₂ receptor agonist or antagonist.

Beneficial effects of modulating PGD₂ receptor activity include achieving one or more of the following in a patient: the treatment or prevention of an inflammatory disease such as asthma, treatment or prevention of allergic rhinitis or arthritis; and the treatment or prevention of a sleep disorder. A patient is a mammal, preferably a human. Reference to patient does not necessarily indicate the presence of a disease or disorder. The term patient includes subjects treated prophylactically and subjects afflicted with a disease or disorder.

Selective agonists or antagonists that mimic or block PGD₂ actions at the DP receptor, CRTH2 and/or FP receptor may have utility in the treatment of disease states or diseases including but not limited to allergic rhinitis and other allergic conditions in which mast cells, eosinophils, TH2 cells and other immune cells express the DP receptor, CRTH2, and/or FP receptor, or produce PGD₂. Additional examples of therapeutic applications include one or more of the following: sleep disorders; glaucoma; osteoporosis; modulators may be useful as cytoprotective, analgesic or anti-inflammatory agents; modulators inhibiting platelet aggregation may be useful for treating vascular disease, prevention of post-injury blood clotting, rejection in organ transplant and by-pass surgery, congestive heart failure, pulmonary hypotension and Raynaud's disease.

Eosinophil PGD₂ Receptor and Inflammation

Eosinophils were found to express the DP receptor and CRTH2. The different effects mediated by PGD₂ at these receptors appear to assist the inflammation response. Pharmacological blockade of PGD₂-mediated events at both the DP receptor and CRTH2 may reduce damage caused by eosinophils at an inflammation site.

In allergic situations, PGD₂ is released by mast cells and may facilitate entry into the inflammation site through DP-mediated vasodilation/extravasation of eosinophils as well as other circulating leukocytes. (Mantovani, et al., Lancet 343:1499, 1994). The entry of eosinophils into the allergic site would be stimulated by the pro-chemokinetic activity of PGD₂ through CRTH2.

The anti-apoptotic and degranulation effects of PGD₂ on eosinophils appear to be playing a factor in inflammation. The survival of resident eosinophils would be prolonged by the anti-apoptotic effect of PGD₂ acting through the DP receptor.

Eosinophil degranulation triggered by PGD₂ activation through CRTH2 causes the release of granule-derived proteins. The effects of granule proteins include cytotoxicity at the bronchial epithelium, an increase in nonspecific bronchial hyperreactivity and impaired ciliary function.

PGD₂ Receptor Assays

Different types of assays formats can be employed making use of the activities identified herein as associated with the eosinophil DP receptor or the eosinophil CRHT2. Examples of such formats include:

(1) Measuring the ability of a compound to affect apoptosis or degranulation;

(2) Identifying a compound that binds to the human eosinophil CRHT2 and then testing the ability of the compound to affect eosinophil morphology, chemokinesis under conditions distinguishing chemokinesis from chemotactic ability, or degranulation; and

(3) Screening for a CRHT2 antagonist using a CRHT2 agonist and measuring the ability of a test compound to modulate changes in eosinophil morphology, chemokinesis under conditions distinguishing chemokinesis from chemotactic ability, or degranulation produced by the agonist.

Measuring the effect of a compound on apoptosis or degranulation provides an overall measure of the effect of the compound on DP receptor or CRTH2 activity. Measuring apoptosis or degranulation also provides a direct measure on activities that it would be desirable to inhibit.

In an embodiment of the present invention, a binding assay is employed to select for compound binding to a prostaglandin D₂ receptor prior to an apoptosis or degranulation assay. Assays measuring the ability of a compound to bind to a DP receptor or CRTH2, employ a DP receptor or CRTH2 polypeptide comprising a PGD₂ binding site. DP receptor and CRTH2 polypeptides include full-length human receptors and functional derivatives thereof, fragments containing a PGD₂ binding site, and chimeric polypeptides comprising such fragments. A chimeric polypeptide comprising a fragment that binds PGD₂ also contains one or more polypeptide regions not found in a human DP receptor or CRTH2.

Preferably, assays measuring PGD₂ binding employ full length human DP receptor or CRTH2. The human DP receptor is described by Abramovitz, et al. U.S. Pat. No. 5,958,723. Human CRTH2 is described by Nagata, et al., The Journal of Immnunology 162:1278-1286, 1999, and Gen-Bank Accession No. AB00535.

PGD₂ receptor amino acid sequences involved in PGD₂ binding can be identified using labeled PGD₂ and different PGD₂ receptor fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding PGD₂ can be subdivided or mutated to further locate the PGD₂ binding region. Fragments used for binding studies can be generated using recombinant nucleic acid techniques.

Binding assays can be performed using recombinantly produced PGD₂ receptor polypeptides present in different environments. Such environments include, for example, cell extracts and purified cell extracts containing a PGD₂ receptor polypeptide expressed from recombinant nucleic acid or naturally occurring nucleic acid and also include, for example, the use of a purified PGD₂ receptor polypeptide produced by recombinant means or from naturally occurring nucleic acid which is introduced into a different environment.

The ability of a compound to antagonize PGD₂ receptor activity can be evaluated using a PGD₂ agonist able to produce receptor activity and then measuring the ability of one or more test compounds to alter such activity. Agonists that can be employed include those able to stimulate both DP receptor activity and CRHT2 activity and those selective for DP receptor activity or CRHT2 activity. Examples of different types of agonists are PGD₂ which acts at both the DP receptor and CRHT2; 13-14-dihydro-15-keto-PGD₂ which is specific for CRTH2; and BW245C which is specific for the DP receptor.

The effectiveness of an antagonist to alter PGD₂ receptor activity can be evaluated by comparing PGD₂ receptor activity in the presence of the agonist with such activity in the presence of the agonist and antagonist. Different types of assay formats can be employed. For example, a control experiment involving an agonist and a test experiment involving the agonist and a test compound can be performed at the same or at different times.

Techniques for measuring apoptosis, morphology, chemokinesis under conditions distinguishing chemokinesis from chemotactic ability, and degranulation are well known in the art. Changes in morphology can be measured visually with the aid of a microscope, such as by scoring cells with irregular shapes. Techniques for measuring morphology include those described in the Examples provided below.

Apoptosis is a type of cell death that is programmed by the cell. Techniques for measuring apoptosis include those described in the Examples provided below.

Chemokinesis is an increase in cell mobility that is brought about by a reagent in the absence of chemical gradient. Techniques for measuring chemokinesis include those described in the Examples provided below.

Degranulation results in the release of granule-derived proteins, such as the major basic protein, the eosinophil cationic protein, eosinophil-derived neurotoxin, and eosinophil peroxidase. Techniques for measuring degranulation include those described in the Examples provided below.

Dosing For Therapeutic Applications

Guidelines for pharmaceutical administration in general are provided in, for example, Remington's Pharmaceutical Sciences 18^{th} Edition, Ed. Gennaro, Mack Publishing, 1990, and Modern Pharmaceutics 2^{nd} Edition, Eds. Banker and Rhodes, Marcel Dekker, Inc., 1990, both of which are hereby incorporated by reference herein.

PGD₂ receptor active compounds having appropriate functional groups can be prepared as acidic or base salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

PGD₂ receptor active compounds can be administered using different routes including oral, nasal, by injection, and transmucosally. Active ingredients to be administered orally as a suspension can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

When administered by nasal aerosol or inhalation, compositions can be prepared according to techniques well known in the art of pharmaceutical formulation. Such techniques can involve preparing solutions in saline, employing benzyl alcohol or other suitable preservatives, absorption promoters to enhance bioavailability, fluorocarbons, or other solubilizing or dispersing agents.

Routes of administration include intravenous (both bolus and infusion), intraperitoneal, subcutaneous, topical with or without occlusion, and intramuscular. Injectable solutions or suspensions known in the art include suitable non-toxic, parenterally-acceptable diluents or solvents, such as mannitol, 1,3-butanediol, water, Ringer's solution and isotonic sodium chloride solution. Dispersing or wetting and suspending agents, include sterile, bland, fixed oils, such as synthetic mono- or diglycerides; and fatty acids, such as oleic acid.

Rectal administration in the form of suppositories, include the use of a suitable non-irritating excipient, such as cocoa butter, synthetic glyceride esters or polyethylene glycols. These excipients are solid at ordinary temperatures, but liquidify and/or dissolve in the rectal cavity to release the drug.

Suitable dosing regimens for therapeutic applications can be obtained taking into account factors well known in the art including age, weight, sex and medical condition of the patient; the severity of the condition to be treated; the route of administration; the renal and hepatic function of the patient; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. The daily dose for a patient is expected to be between 0.01 and 1,000 mg per adult patient per day.

### EXAMPLES

Examples are provided below to further illustrate different features of the present invention. The examples also illustrate useful methodology for practicing the invention. These examples do not limit the claimed invention.

### Example 1

### Material and Methods

This example illustrates different reagents and techniques.

Reagents

PGD₂, fluprostenol and PGE₂ were obtained from Biomol Research Laboratories, (Plymouth Meeting, Pa.). BW245C, 13,14-dihydro-15-keto-PGD₂ (DK-PGD₂) and latanoprost (free acid) were from Cayman Chemical (Ann Arbor, Mich.). Platelet activating factor (PAF) was from Sigma (St-Louis, Mo.). Recombinant human interleukin-5 was produced using a baculovirus system and purified by FPLC. (Brown, et al., Protein Expr. Purif. 6:63, 1995.)

Eosinophil Purification

Circulating eosinophils were isolated from heparinized venous blood from normal volunteers. Erythrocytes were removed by addition of Dextran to a final concentration of 0.9% (Dextran T500 from Pharmacia prepared as a 6% stock in 0.9% saline solution). After a 45 minute incubation at room temperature, the leukocytes in the plasma fraction were collected by centrifugation (4° C., 300× g, 10 minutes), and resuspended in Hank's balanced salt solution (HBSS without calcium and magnesium).

A density step gradient was generated by placing 20 ml of Ficoll-Paque™ (Pharmacia) under 30 ml of resuspended cells. The gradient was centrifuged (4° C., 400× g, 30 minutes) and the pellet containing the granulocytes was resuspended in 10 ml of water for 15 seconds to lyse any residual erythrocytes. The hypotonic lysis was stopped by the addition of 40 ml of HBSS.

The cells were then centrifuged (4° C., 300× g, 10 minutes), washed once with 50 ml of HBSS and resuspended in Dulbecco phosphate buffer saline (PBS without calcium and magnesium from GIBCO-BRL) at a concentration of 1×10⁹ cells per ml. An equal volume of CD16 magnetic beads (Milteny Biotec) was added and incubated at 4° C. for 30 minutes. At the end of the incubation, the volume was brought to 1 ml with PBS (without Ca⁺² and Mg⁺²) and applied to a CS separation column placed in the magnetic field of a MACS separator (Milteny Biotec).

The CD16+ neutrophils were retained in the column while a >95% pure fraction of CD16-eosinophils eluted from the column. The purity of the eosinophil fraction was evaluated by flow cytometry (CELL-DYN 3700 System) based on size, complexity, granularity and lobularity.

RT-PCR and Southern Blot

Total RNA was obtained from isolated eosinophils (>95% pure) by using a total RNA isolation kit (Rneasy kit, Qiagen) and treated with DNAse (Gibco-BRL) prior to reverse transcription (Gene Amp kit, Perkin Elmer). Amplification of DP receptor by PCR (Advantage GC kit, Clontech) used the following primers: DP sense, 5′-ACAACTCGTTGTGCCAAGCC (SEQ. ID. NO. 1); DP antisense, 5′-GCATCGCATAGAGGTTGCGC (SEQ. ID. NO. 2); CRTH2 sense, 5′-CTACAATGTGCTGCTCCTGAAC (SEQ. ID. NO. 3); CRTH2 antisense, 5′-CAGGTGAGCACGTAGAGCAC (SEQ. ID. NO. 4). The PCR reaction (50 μl). included a denaturation step (94° C., 1 minute) and 35 cycles of PCR (94° C., 30 seconds; 55° C., 30 seconds; 68° C., 1 minutes).

PCR reactions were electrophoresed in agarose gels and transferred to nylon N+Hybond membrane (Amersham). The blot was hybridized with a ³²P-labeled DNA fragment encoding the full-length hCRTH2 or hDP receptor in ExpressHyb solution (Clontech) overnight at 68 ° C. The blot was washed twice in 2×SSC (at 65° C.) and twice in 0.2×SSC (at 65° C.) for 30 minutes each. Results were revealed by autoradiography.

In Situ Hybridization

Freshly isolated eosinophils (2×10⁵) were layered onto a poly-D-lysine coated glass slide by centrifugation (Cytospin). Cells were then fixed in 4% paraformaldehyde solution prepared in PBS (pH of 7.4) for 20 minutes at room temperature. The slides were then processed as follows: 2 minutes in 3× PBS, 2 times 2 minutes in 1× PBS and incubations of 5 minutes in 50%, 70%, 95% and 100% aqueous ethanol solutions. Slides were air dried and stored at −80° C.

The slides were thawed to room temperature and washed for 5 minutes in diethylpyrocarbonate (DEPC)-treated water and twice in PBS. The sections were treated with 1.0 μg/ml proteinase K in 100 mM Tris, pH 8.0, 50 mM EDTA for 10 minutes at 37° C. and washed for 5 minutes in DEPC-treated water. The slides were then washed in 0.1 M triethanolamine, pH 8.0 (TEA) for 5 minutes and washed again for 10 minutes in TEA with 0.25% acetic anhydride. Finally, the sections were washed twice for 5 minutes in 2×SSC.

A 398 bp fragment representing the 5′ terminal end of the human DP receptor cDNA was amplified by PCR and subcloned into the PCR II dual promoter vector (Invitrogen). The plasmid was linearized using either Xho I or Spe I and digoxigenin-labeled (DIG) riboprobes were synthesized using the DIG-RNA labeling kit from Boehringer Mannheim. The riboprobes were diluted in 75% hybridization buffer (75% formamide, 3×SSC, 1× Denhardt's, 0.2 mg/ml yeast tRNA, 50 mM sodium phosphate, 10% dextran sulfate) and layered onto the cytospin slides. The slides were covered with parafilm and left to hybridize for 16 hours at 55° C. in a humidified (75% formamide) chamber. The parafilm was then removed by soaking the slides in 2×SSC for 30 minutes. The sections were then treated with RNase A (40 μg/ml in 10 mM Tris, pH 8.0, 500 mM NaCl) for 45 minutes at 37° C. The slides were washed in 2×SSC, 1×SSC, 0.5×SSC (for 10 minutes each at room temperature) and 0.1×SSC (45 minutes at 60° C.).

Colorimetric detection of the DIG-labeled riboprobe was done using an alkaline phosphatase-linked anti-DIG antibody (Boehringer Mannheim). All subsequent steps were carried out at room temperature. The slides were washed in detection buffer (DB; 100 mM Tris pH 7.5, 150 mM NaCl, 0.1% Tween), incubated with SuperBlock buffer (Biogenex) for 10 minutes and then incubated for 2 hours with the antibody (1:75 dilution) in DB and then washed three times in DB. The chromagen solution (Fast Red, Sigma biochemicals) was then added and the slides were left to incubate for 30 minutes. The reaction was stopped by washing in 10 mM Tris pH 8.0, 1 mM EDTA. The cells were mounted using SlowFade (Molecular probes) and examined on a fluorescent microscope connected to a CCD camera.

Microscopy

Purified eosinophils were incubated in RPMI 1640 media supplemented with 0.5% fetal bovine serum in the presence of the compound to be tested for 15 minutes in a 24-well dish. Light microscopy was performed with an inverted Axiovert 25 (Zeiss) and images were obtained with a 35 mm SLR camera (ARIA CONTAX, Kyocera corporation) using Kodak Elite Chrome 160T film.

Eosinophil Chemokinesis

Purified eosinophils were resuspended at 3.0×10⁶ cells per ml in RPMI 1640 medium supplemented with 0.5% (v/v) fetal bovine serum. Compounds to be tested were added from 1000× concentrated stock solutions to 100 μl of cells in a 1.5 ml centrifuge tube and incubated at room temperature for 5 minutes. 100 μl of treated cells were then added to the top half of a chemotactic chamber (6.5 mm Transwell, 3.0 μm polycarbonate membrane from Costar) and 600 μl of RPMI, supplemented with 0.5% (v/v) fetal bovine serum, was added to the bottom chamber. After a 2 hour incubation at 37° C. in a CO₂ chamber, the top chamber was discarded and the number of cells that had migrated to the lower chamber was evaluated by counting the cells using an hematocytometer. For each condition tested, the number of migrating eosinophils in two chemotactic chambers was averaged.

Eosinophil Degranulation

Purified eosinophils were resuspended at 3.0×10⁶ cells per ml in RPMI 1640 medium supplemented with 0.5% (v/v) fetal bovine serum. Compounds to be tested were diluted 1:1000 to their final concentration in 300 μl of cells in a 1.5 ml centrifuge tube. The cells were immediately transferred to a 24-well plate. After an 1 hour incubation at 37° C. in a CO₂ chamber, the cells were removed by centrifugation (4° C., 300× g, 10 minutes). Eosinophil cationic protein (ECP) in the supernatant was quantified by a double antibody radioimmunoassay (Pharmacia) following the manufacturer's protocol.

In Vitro Eosinophil Apoptosis Assay

Purified eosinophils were resuspended at 2.0×10⁵ cells per ml in RPMI 1640 medium supplemented with 10% (v/v) fetal bovine serum, 2 mM glutamine, and 100 units of penicillin and streptomycin. Compounds to be tested were diluted 1:1000 to their final concentration and the cells were incubated at 37° C. in a CO₂ chamber for 36 hours. The extent of apoptosis in the eosinophil population was evaluated using the TACS™ Annexin-V-FITC apoptosis detection kit (R&D systems). Non-apoptotic cells are not stained with either Annexin-V FITC or propidium iodide. Early apoptotic cells are stained with Annexin-V FITC but not propidium iodide (green fluorescence). Late apoptotic cells are stained with both Annexin-V FITC and propidium iodide (dual green and red fluorescence). Necrotic cells are only stained with propidium iodide (red fluoresecence). Labeled eosinophils were analyzed in a FACS Calibur system from Becton Dickinson.

### Example 2

### Eosinophil Expression of DP and CRTH2

To establish which PGD₂-binding receptors are expressed by human eosinophils, RT-PCR was performed on total RNA from human eosinophil (>95% purity). The identity of the PCR products was confirmed by Southern blot detection using DP receptor and CRTH2-specific probes.

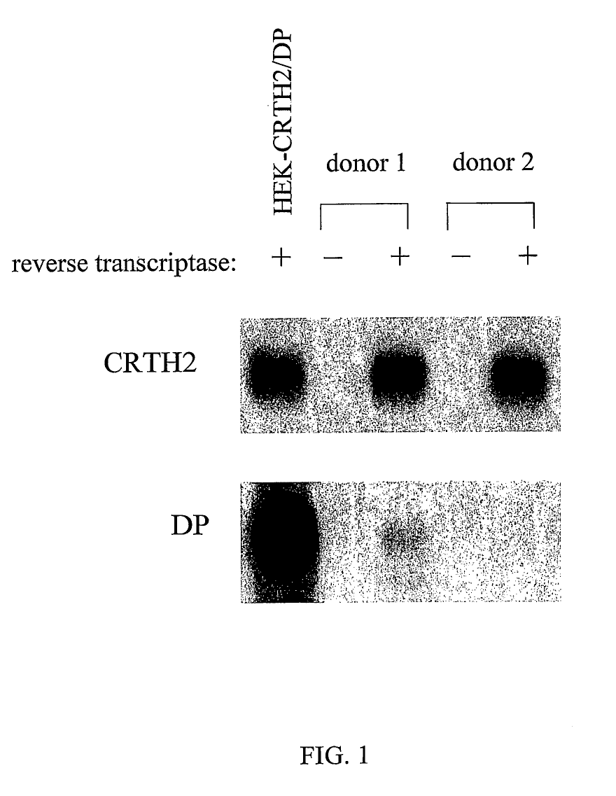

CRTH2 mRNA was detected in eosinophils from four donors while DP mRNA was detected in only two of the four donors (FIG. 1). The identity of the cell type expressing DP receptor as an eosinophil was confirmed by in situ hybridization. DP antisense hybridized only to cells showing the characteristic bi-lobal nucleus of eosinophils.

### Example 3

### PGD₂ Induced a Change in Eosinophil Morphology Through CRTH2

PGD₂ (<10 nM) induced dramatic changes in cell morphology within minutes. Vehicle-treated eosinophils were spherical and only weakly adhered to the culture dish. In contrast, eosinophils treated with PGD₂ become flat, assumed an Amoeba-like shape and showed round structures which may represent secretory vesicles (panel 2b). This effect was seen on the majority of eosinophils from all donors analysed (n=20). PGD₂-treated eosinophils reverted to a spherical shape within six hours. These cells were resistant to morphology changes after a second PGD₂ challenge (data not shown).

The DP receptor selective agonist, BW245C, at concentrations as high as 1 μM did not affect eosinophil shape (panel 2c). In contrast, a CRTH2 selective agonist, DK-PGD₂, induced a morphological change identical to that observed with PGD₂ (panel 2d). Known activators of eosinophils such as platelet activating factor (PAF) (panel 2e) as well as interleukin-5 (Il-5) (panel 2f) also lead to a rapid change in eosinophil morphology. Other prostanoid receptor agonists such as PGE₂ (EP receptors) as well as fluprostenol and latanoprost (FP receptor) did not cause, even at μM doses, any alterations of eosinophil morphology (data not shown). These data suggest that the morphological changes induced by PGD₂ and DK-PGD₂ on eosinophils are mediated through the CRTH2 and not the DP receptor.

### Example 4

### PGD₂ Increases Eosinophil Chemokinesis Through CRTH2

PGD₂ increased cell motility in the absence of a chemical gradient, a process defined as chemokinesis. PGD₂ was not observed to exert a chemotatic effect. Overall, the data indicates that PGD₂ modulates eosinophil chemokinesis in a DP-independent manner and most likely through the CRTH2.

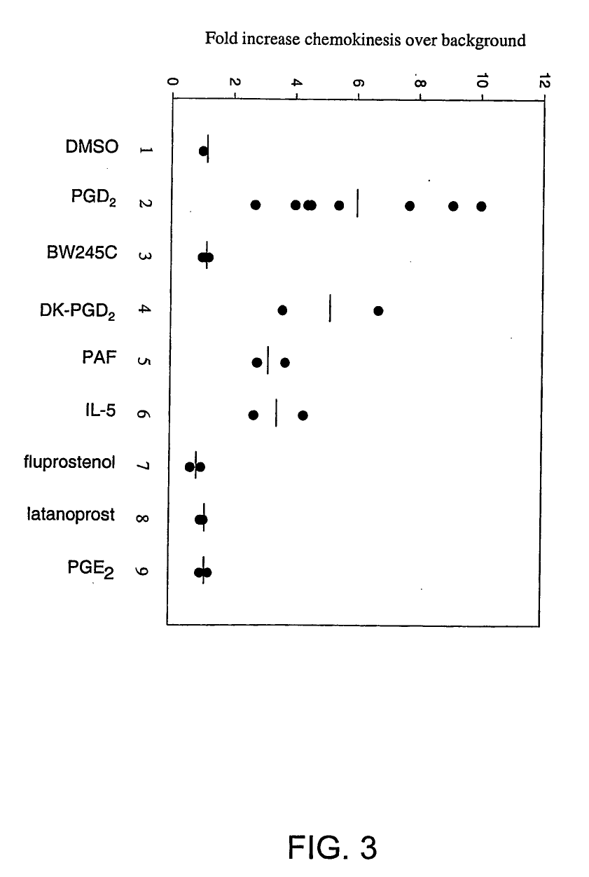

Chemokinesis was measured by incubating eosinophils with PGD₂ for 5 minutes prior to their loading in the upper chamber of a chemotactic unit lacking a chemoattractant in the lower chamber. PGD₂ at a concentration of 100 mM increased eosinophil chemokinesis by 6-fold compared to cells treated with vehicle only (FIG. 3). PGD₂ at concentrations of 10 nM and 1 μM increased eosinophil chemokinesis by a factor of 5 and 9-fold respectively (data not shown). PAF and Il-5 were also able to stimulate eosinophil chemokinesis (FIG. 3). (See, Wardlaw, et al., J. Clin. Invest. 78:1701, 1986, Schweizer, et al., J. Leukoc. Biol. 59:347, 1996.) DK-PGD₂ but not BW245C was effective in stimulating eosinophil chemokinesis (FIG. 3). EP and FP receptor agonists, PGE₂, fluprostenol and latanoprost failed to modulate eosinophil migration.

Chemotaxis was measured by adding PGD₂ to the bottom chamber of a chemotactic unit containing eosinophils in the top chamber. In contrast to chemoattractants such as PAF and eotaxin, PGD₂ was not a chemoattractant since it did not attract eosinophils to the lower chamber of the chemotactic unit (data not shown). Eosinophils pre-incubated with PGD₂ (up to 1 μM for 5 minutes to 18 hours) did not have an altered chemotactic response to either PAF or eotaxin (data not shown).

### Example 5

### PGD₂ Triggers Eosinophil Degranulation Through CRTH2

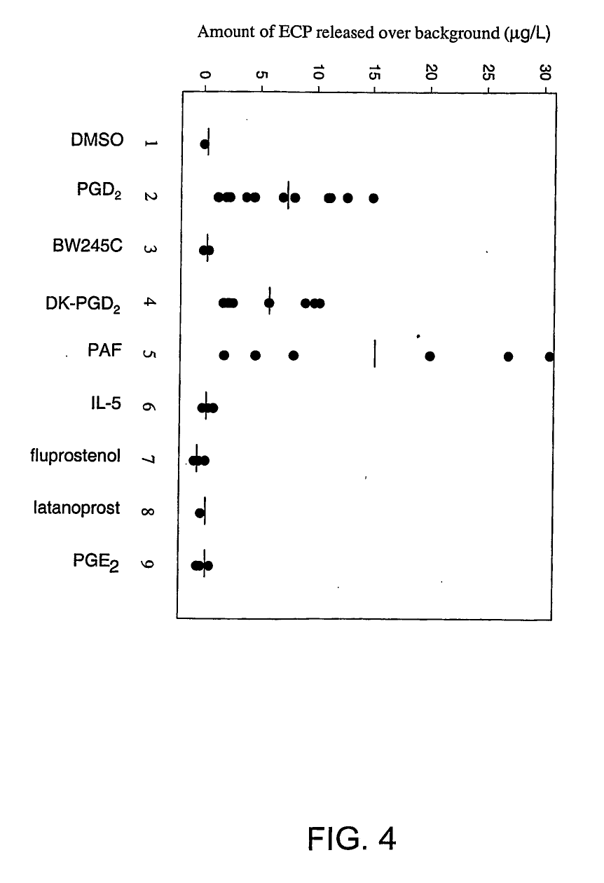

Degranulation of eosinophils was assayed by challenging freshly isolated eosinophils with PGD₂ and measuring release of the eosinophil cationic protein (ECP) into the media using an ECP-specific radioimmunoassay. PGD₂ at a concentration of 10 to 100 nM significantly increased the release of ECP from eosinophils into the media (FIG. 4).

Among the donors, a broad range in PAF- and PGD₂-induced release of ECP is seen. On average, the extent of PGD₂-induced ECP release is about half that seen with PAF. In general the extent of degranulation induced by PGD₂ and PAF paralleled each other. ECP was not released as a result of necrosis as lactate dehydrogenase (LDH), a marker for necrotic cell lysis, was not detected in the media (data not shown). We also observed the release from eosinophils of another granular protein, EDN, after PGD₂ challenge (data not shown). DK-PGD₂, but not BW245C, significantly increased the release of ECP (FIG. 4). FP and EP receptor agonists did not induce ECP release. As seen with eosinophil morphology changes and chemokinesis, PGD₂ induces eosinophil degranulation by a CRTH2-dependent mechanism.

### Example 6

### A Selective DP Agonist Delays the Onset of Apoptosis

The ability of PGD₂ to modulate apoptosis in eosinophils was measured by quantifying the capacity of Annexin V to bind to phosphatidylserine on the outer membrane of apoptotic cells. (Koopman, et al., Blood 84:1415, 1994.) Necrotic cell death was determined by propidium iodide uptake. (Darzynkiewicz, et al., Cytometry 13:795, 1992.) Annexin V-FITC and propidium iodide staining of eosinophils was evaluated by FACS analysis.

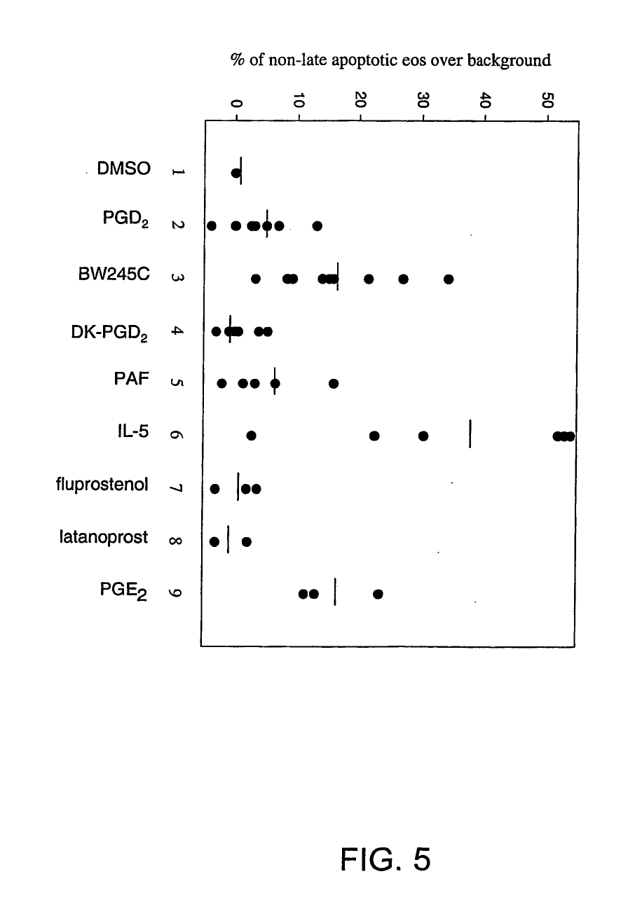

Isolated eosinophils become apoptotic after approximately 12 hours when cultured in RPMI-1640 supplemented with 10% fetal bovine serum. After 48 hours almost all eosinophils were dead (data not shown). Addition of Il-5 or PGE₂ to the media increased the percentage of non-apoptotic eosinophils at 36 hours in culture (FIG. 5).

PGD₂ was a weak inhibitor of apoptotic cell death while DK-PGD₂ had no significant effect (FIG. 5). In contrast, the DP-specific agonist BW245C significantly increased the percentage of non-apoptotic eosinophil by 17%. The effects of FP agonists, fluprostenol and latanoprost were not significant.

Other embodiments are within the following claims. While several embodiments have been shown and described, various modifications may be made without departing from the spirit and scope of the present invention.

What is claimed is:

1. A method of assaying the ability of a test compound to modulate prostaglandin D₂ receptor activity comprising the steps of:
a) providing said test compound to an eosinophil; and
b) measuring the effect of said test compound on either apoptosis or degranulation as a measure of the ability of said test compound to modulate prostaglandin D₂ receptor activity.

2. The method of claim 1, wherein prior to said step (a) said test compound has been identified as able to bind to the DP receptor and said method measures apoptosis.

3. The method of claim 1, wherein said method further comprises providing a DP receptor agonist to said eosinophil and measuring the ability of said test compound to affect apoptosis.

4. The method of claim 1, wherein prior to said step (a) said test compound has been identified as able to bind to CRTH2 and said method measures degranulation.

5. A method of assaying the ability of a test compound to modulate CRTH2 receptor activity comprising the steps of:
a) identifying a compound that binds to a human CRTH2;
b) providing said test compound to an eosinophil; and
c) measuring either eosinophil morphology, chemokinesis under conditions distinguishing chemokinesis from chemotactic ability, or degranulation.

6. The method of claim 5, wherein said step (c) measures eosinophil morphology.

7. The method of claim 5, wherein said step (c) measures chemokinesis.

8. The method of claim 5, wherein said step (c) measures degranulation.

9. A method of assaying the ability of a test compound to modulate CRTH2 activity comprising the steps of:
a) providing said test compound and an CRTH2 agonist to an eosinophil; and
b) measuring either eosinophil morphology, chemokinesis under conditions distinguishing chemokinesis from chemotactic ability, or degranulation.

10. The method of claim 9, wherein said step (c) measures eosinophil morphology.

11. The method of claim 9, wherein said step (c) measures chemokinesis.

12. The method of claim 9, wherein said step (c) measures degranulation.

13. The method of claim 12, wherein said agonist distinguishes CRTH2 from the DP receptor.

14. The method of claim 13, wherein said agonist is 13-14-dihydro-15-keto-PGD₂.
